(19) United States
(12) Patent Application Publication
(10) Pub. No.: US 2023/0411018 A1
(43) Pub. Date: Dec. 21, 2023

(54) METHOD AND APPARATUS FOR PREDICTING OCCURRENCE OF DISEASE

(71) Applicant: ONTACT HEALTH CO., LTD., Seoul (KR)

(72) Inventors: Su Jin LEE, Seoul (KR); Hack Joon SHIM, Seoul (KR); Ji Min SUNG, Seoul (KR); Young Taek HONG, Seoul (KR); Ga Eun KIM, Seoul (KR); Seong Min HA, Seoul (KR); Shin Hee MAENG, Seoul (KR)

(21) Appl. No.: 18/251,594

(22) PCT Filed: Oct. 20, 2021
(86) PCT No.: PCT/KR2021/014754

§ 371 Date: May 3, 2023

(30) Foreign Application Priority Data
Nov. 4, 2020 (KR) 10-2020-0145947
Sep. 16, 2021 (KR) 10-2021-0123951

(51) Int. Cl. G16H 50/30; G16H 50/70; G16H 10/60

(52) U.S. Cl. CPC G16H 50/30; G16H 50/70; G16H 10/60

(57) ABSTRACT

The present invention relates to predicting a future onset possibility of a disease by using an artificial intelligence algorithm, and a method for predicting the onset of the disease may include: obtaining input data based on medical checkup data of a subject; generating output data indicating an onset possibility of the disease by year from the input data by using a trained artificial intelligence model; determining at least one item with a relatively high contribution to a result of the output data; and outputting information regarding the onset possibility of the disease by year and the at least one item.

### TECHNICAL FIELD

The present invention relates to predicting the onset of a disease and, more particularly, to a method and device for predicting a future onset possibility of the disease by using an artificial intelligence (AI) algorithm.

### BACKGROUND ART

Diseases are a condition that causes a disorder and thus impedes the normal function of a human mind or body, and depending on the seriousness of diseases, man undergoes sufferings and even end their lives. Accordingly, over the course of human history, a variety of social systems and technologies have been developed to diagnose, treat and even prevent diseases. For diagnosis and treatment of diseases, various tools and methods have been devised along with impressive technical advances, but the final judgments are still dependent on doctors.

Meanwhile, the recent advancement of artificial intelligence (AI) technology is so remarkable as to draw attention from various fields. In particular, massive accumulations of medical data and the image-centered data environment encourage various attempts and studies to graft AI algorithms onto medicine. Specifically, various studies are using AI algorithms to provide solutions to the diagnosis and prediction of diseases and other tasks that still depend on clinical judgments.

### DISCLOSURE

### Technical Problem

The present invention is directed to provide a method and device for effectively predicting a future onset possibility of a disease for a subject.

The present invention is directed to provide a method and device for predicting a disease onset possibility on an annual basis for a predetermined period.

The present invention is directed to provide a method and device for determining a contributed factor affecting determination of a disease onset possibility.

The present invention is directed to provide a method and device for more accurately predicting a risk of outbreak at a specific time by considering a time interval between multiple times when there is health data corresponding to the multiple times for a person.

It is to be understood that the technical objects to be achieved by the present invention are not limited to the aforementioned technical objects, and other technical objects not mentioned herein will be apparent to those of ordinary skill in the art to which the present invention pertains from the following description.

### Technical Solution

A method for predicting the onset of a disease according to an embodiment of the present invention may include: obtaining input data based on medical checkup data of a subject; generating output data indicating an onset possibility of the disease by year from the input data by using a trained artificial intelligence model; determining at least one item with a relatively high contribution to a result of the output data; and outputting information regarding the onset possibility of the disease by year and the at least one item.

According to an embodiment of the present invention, the artificial intelligence model may be trained by using learning data based on medical checkup data of at least one examinee diagnosed positive for the disease and at least one examinee diagnosed negative for the disease, and the learning data may include basic learning data generated based on the medical checkup data and augmented learning data generated based on data derived from the medical checkup data.

According to an embodiment of the present invention, the derived data may include data sets corresponding to a plurality of subsets for times of performing medical checkup included in the medical checkup data.

According to an embodiment of the present invention, the learning data may include a plurality of data sets, each of the plurality of data sets may include checkup result information of a first time, time difference information between a second time of performing the medical checkup immediately before the first time and the first time, and label data based on disease diagnosis time information of a corresponding examiner, and the label data may have a vector form indicating whether or not the disease occurs per a unit time that equally divides a predefined period.

According to an embodiment of the present invention, the time difference information may be set to 0, when the first time is an earliest time of performing the medical checkup.

According to an embodiment of the present invention, the artificial intelligence model may be configured to receive, as input, checkup result information of a subject for each time of a plurality of times and a time interval value from a previous time corresponding to each piece of checkup result information, to recurrently generate a hidden state value by considering the time interval value, and to generate, as output, an onset possibility value of the disease per the unit time, which equally divides the predefined period, based on a final hidden state value that is generated by a predetermined number of cycles.

According to an embodiment of the present invention, the artificial intelligence model may include a network that generates output data in a form including as many onset possibility values of the disease as the number of unit times equally dividing the predefined period based on the final hidden state value.

According to an embodiment of the present invention, the determining of the at least one item may include: determining a relevance score of each node sequentially from an output layer to an input layer of the artificial intelligence model; selecting at least one node among nodes in the input layer based on relevance scores of the nodes; and checking at least one diagnosis item corresponding to the at least one selected node.

A method of predicting a disease according to an embodiment of the present invention may include: obtaining, by a communication unit, health data of a person and comparison information from an external device, wherein the health data includes health data of multiple times for the person and a time interval between the multiple times; and producing, by a processor, disease prediction information by using a long short-term memory (LSTM) based on the health data including the time interval and comparison information.

According to an embodiment of the present invention, the producing of the disease prediction information may produce the disease prediction information at a preset future time interval from a present time.

According to an embodiment of the present invention, the producing of the disease prediction information may generate numerical information quantifying an onset probability for a corresponding disease and, when the numerical information is equal to or greater than a preset threshold, determine that the disease occurs.

According to an embodiment of the present invention, the producing of the disease prediction information may generate the numerical information for a corresponding disease at a preset future time interval from a present time and, when the numerical information is equal to or greater than a preset threshold at a first time, even if the numerical information is less than the preset threshold at a second time that is later than the first time, determine that the disease also occurs at the second time.

According to an embodiment of the present invention, the comparison information includes comparison information of multiple times and a time interval between the multiple times, and the producing of the disease prediction information may produce the disease prediction information based on the health data including the time interval and the comparison information including the time interval.

According to an embodiment of the present invention, the at least one item may be selected from items that are subject to modification in future.

A method for predicting the onset of a disease according to an embodiment of the present invention may include: obtaining input data based on medical checkup data of a subject; and providing output data indicating an onset possibility of the disease by year from the input data by using a trained artificial intelligence model, and the artificial intelligence model may be trained based on checkup result information of medical checkups performed at an unequal time interval, and the output data may include onset possibility values of the disease per a unit time that equally divides a predefined period.

A program stored in a medium according to an embodiment of the present invention may implement the above-described method, when being operated by a processor.

A device for predicting the onset of a disease according to an embodiment of the present invention may include: a transceiver; a storage unit configured to storing an artificial intelligence model; and at least one processor coupled to the transceiver and the storage unit, and the at least one processor may further be configured to: obtain input data based on medical checkup data of a subject, generate output data indicating an onset possibility of the disease by year from the input data by using a trained artificial intelligence model, determine at least one item with a relatively high contribution to a result of the output data, and output information regarding the onset possibility of the disease by year and the at least one item.

A device for predicting the onset of a disease according to an embodiment of the present invention may include: a transceiver; a storage unit configured to storing an artificial intelligence model; and at least one processor coupled to the transceiver and the storage unit, and the at least one processor may further be configured to obtain input data based on medical checkup data of a subject and to provide output data indicating an onset possibility of the disease by year from the input data by using a trained artificial intelligence model, and the artificial intelligence model may be trained based on checkup result information of medical checkups performed at an unequal time interval, and the output data may include onset possibility values of the disease per a unit time that equally divides a predefined period.

A disease prediction system according to another embodiment of the present invention may include: a communication unit configured to obtain health data of a person and comparison information from an external device, wherein the health data includes health data of multiple times for the person and a time interval between the multiple times; and a processor configured to produce disease prediction information by using a long short-term memory (LSTM) based on the health data including the time interval and comparison information.

According to an embodiment of the present invention, the processor may be configured to produce the disease prediction information at a preset future time interval from a present time.

According to an embodiment of the present invention, the processor may further be configured to generate numerical information quantifying an onset probability for a corresponding disease and, when the numerical information is equal to or greater than a preset threshold, determine that the disease occurs.

According to an embodiment of the present invention, the processor may further be configured to generate the numerical information for a corresponding disease at a preset future time interval from a present time and, when the numerical information is equal to or greater than a preset threshold at a first time, even if the numerical information is less than the preset threshold at a second time that is later than the first time, determine that the disease also occurs at the second time.

According to an embodiment of the present invention, the comparison information includes comparison information of multiple times and a time interval between the multiple times, and the processor may further be configured to produce the disease prediction information based on the health data including the time interval and the comparison information including the time interval.

The features briefly summarized above for the present invention are only illustrative aspects of the detailed description of the invention that follows, but do not limit the scope of the present invention.

### Advantageous Effects

According to the present invention, a future onset possibility of a disease may be predicted at a predetermined time unit by using a learned artificial intelligence model.

In addition, according to the present invention, when health data corresponding to multiple times exists for a person, there is an advantage that a risk of outbreak is predicted for a specific disease at a specific time by considering every past record of medical checkup.

It is to be understood that effects to be obtained by the present invention are not limited to the aforementioned effects, and other effects not mentioned herein will be apparent to those of ordinary skill in the art to which the present invention pertains from the following description.

### DESCRIPTION OF DRAWINGS

FIG. 1 illustrates a system according to an embodiment of the present invention.

FIG. 2 illustrates a structure of a device for predicting a disease onset possibility according to an embodiment of the present invention.

FIG. 3 illustrates an example of a perceptron constituting an artificial intelligence model applicable to the present invention.

FIG. 4 illustrates an example of an artificial neural network constituting an artificial intelligence model applicable to the present invention.

FIG. 5 illustrates an example of a long short-term memory (LSTM) network applicable to the present invention.

FIG. 6 illustrates an example of data used for predicting a disease onset possibility according to an embodiment of the present invention.

FIG. 7A illustrates an example of a structure of an artificial intelligence model for predicting a disease onset possibility according to an embodiment of the present invention.

FIG. 7B illustrates an example of a structure of a hidden layer of an artificial intelligence model for predicting a disease onset possibility according to an embodiment of the present invention.

FIG. 8 illustrates an example of an output generated by an artificial intelligence model for predicting a disease onset possibility according to an embodiment of the present invention.

FIG. 9 illustrates a forward process for predicting a disease onset possibility and a reverse process for determining a contributed factor in accordance with an embodiment of the present invention.

FIG. 10 illustrates an example of a procedure of training an artificial intelligence model according to an embodiment of the present invention.

FIG. 11 illustrates an example of an augmentation procedure for learning data according to an embodiment of the present invention.

FIG. 12 illustrates an example of a procedure of predicting a disease onset possibility by using an artificial intelligence model according to an embodiment of the present invention.

FIG. 13 illustrates an example of a method of predicting a disease according to an embodiment of the present invention.

FIG. 14 illustrates an example of numerical information for explaining a step of producing disease prediction information in a method of predicting a disease according to an embodiment of the present invention.

### MODE FOR INVENTION

Hereinafter, embodiments of the present invention will be described in detail with reference to the accompanying drawings, which will be easily implemented by those skilled in the art. However, the present invention may be embodied in many different forms and is not limited to the embodiments described herein.

In the following description of the exemplary embodiments of the present invention, a detailed description of known functions and configurations incorporated herein will be omitted when it may make the subject matter of the present invention rather unclear. In addition, parts not related to the description of the present invention in the drawings are omitted, and like parts are denoted by similar reference numerals.

The present invention relates to predicting a disease onset possibility by using an artificial intelligence algorithm and, more particularly, to a technique of training the artificial intelligence model by using data, which is generated irregularly in time, and of predicting the disease onset possibility at a predetermined time unit by using the trained artificial intelligence model.

In addition, the present invention relates to a disease prediction system, a disease prediction method and a recording medium implementing the method and, more particularly, to the disease prediction system, the disease prediction method and the recording medium implementing the method, which predict a disease onset probability at a specific time by using health data of a person.

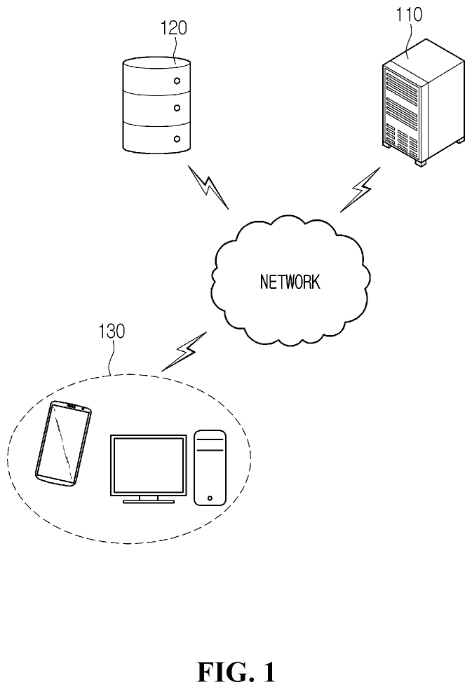

FIG. 1 illustrates a system according to an embodiment of the present invention.

Referring to FIG. 1, a system may include a service server 110, a data server 120, and at least one client device 130.

The service server 110 provides a service based on an artificial intelligence model. That is, the service server 110 performs a learning and prediction operation by using the artificial intelligence model. The service server 110 may perform communication with the data server 120 or the at least one client device 130 via a network. For example, the service server 110 may receive learning data for training the artificial intelligence model from the data server 120 and perform training. The service server 110 may receive data necessary for a learning and prediction operation from the at least one client device 130. In addition, the service server 110 may transmit information on a prediction result to the at least one client device 130.

The data server 120 provides learning data for training of an artificial intelligence model stored in the service server 110. According to various embodiments, the data server 120 may provide public data accessible to anyone or data requiring permission. When necessary, learning data may be preprocessed by the data server 120 or the service server 120. According to another embodiment, the data server 120 may be omitted. In this case, the service server 110 may use an artificial intelligence model that is externally trained, or learning data may be provided offline to the service server 110.

The at least one client device 130 transmits and receives data associated with an artificial intelligence model, which is managed by the service server 110, to and from the service server 110, respectively. The at least one client device 130 may be an equipment used by a user, transmits information input by the user to the service server 110, and store or provide (e.g., mark) information received from the service server 110 to the user. According to a situation, a prediction operation is performed based on data transmitted from any one client, and information associated with a result of prediction may be provided to another client. The at least one client device 130 may be a computing device with various forms like a desktop computer, a laptop computer, a smartphone, a tablet PC, and a wearable device.

Although not illustrated in FIG. 1, the system may further include a management device for managing the service server 110. Being a device used by a subject that manages a service, the management device monitors a state of the service server 110 or controls a setting of the service server 110. The management device may access the service server 110 via a network or be directly connected with the service server 110 through a cable connection. According to a control of the management device, the service server 110 may set a parameter for operation.

As described with reference to FIG. 1, the service server 110, the data server 120, the at least one client device 130, and a management device may be connected via a network and interact with each other. Herein, the network may include at least one of a wired network and a wireless network and consist of any one of a cellular network, a short-range network, and a wide area network or a combination of two or more thereof. For example, the network may be embodied based on at least one of a local area network (LAN), a wireless LAN (WLAN), Bluetooth, LTE (long term evolution), LTE-A (LTE-advanced), and 5G (5th generation).

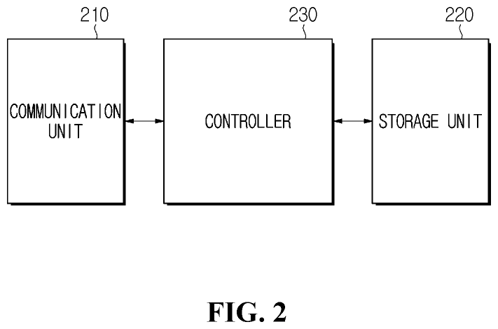

FIG. 2 illustrates a structure of a device for predicting a disease onset possibility according to an embodiment of the present invention. The structure exemplified in FIG. 2 may be understood as a structure of the service server 110, the data server 120, and the at least client device 130 of FIG. 1.

Referring to FIG. 2, a device includes a communication unit 210, a storage unit 220, and a controller 230.

The communication unit 210 accesses a network and performs a function for communicating with another device. The communication unit 210 supports at least one of wired communication and wireless communication. For communication, the communication unit 210 may include at least one of a radio frequency (RF) processing circuit and a digital data processing circuit. According to a case, the communication unit 210 may be understood as a component including a terminal for connecting a cable. Since the communication unit 210 is a component for transmitting and receiving data and a signal, the communication unit 210 may be referred to as ‘transceiver’.

The storage unit 220 stores data, a program, a micro code, a set of instructions, and an application, which are necessary to operate a device. The storage unit 220 may be embodied as a temporary or non-temporary storing medium. In addition, the storage unit 210 may be embodied in a fixed form in a device or in a separable form. For example, the storage unit 220 may be embodied in at least one of a NAND flash memory such as a compact flash (CF) card, a secure digital (SD) card, a memory stick, a solid-state drive (SSD) and a micro SD card and a magnetic computer memory device like a hard disk drive (HDD).

The controller 230 controls an overall operation of a device. To this end, the controller 230 may include at least one processor and at least one microprocessor. The controller 230 may execute a program stored in the storage unit 220 and access a network via the communication unit 210. Particularly, the controller 230 may execute algorithms according to various embodiments described below and control a device to operate according to the embodiments described below.

Based on a structure described with reference to FIG. 1 and FIG. 2, a service based on an artificial intelligence algorithm may be provided according to various embodiments of the present invention. Herein, an artificial intelligence model consisting of an artificial neural network may be used to implement an artificial intelligence algorithm. The concepts of perceptron, which is a constituent unit of an artificial neural network, and the artificial neural network are as follows.

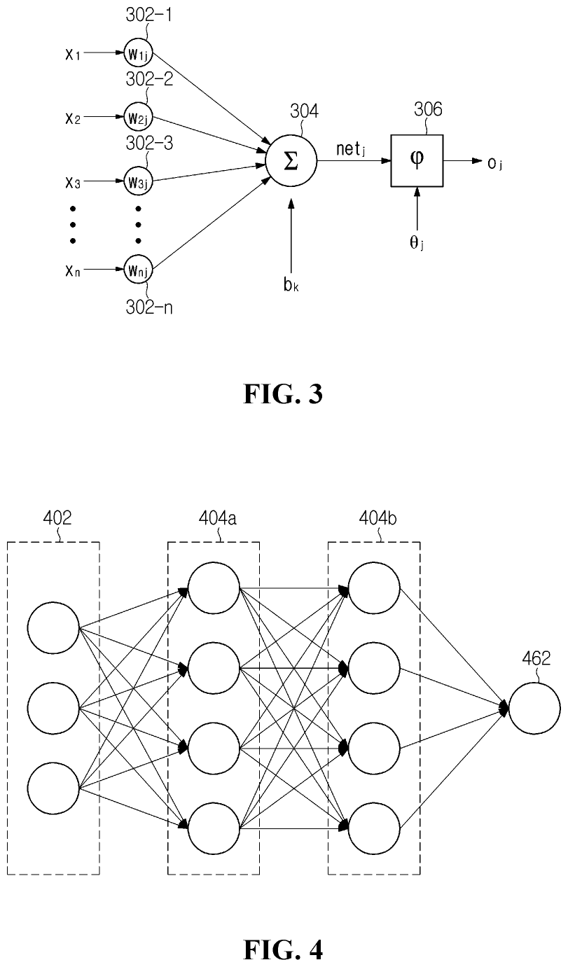

Being modeled after neurons of a living thing, perceptrons have a structure of outputting a single signal from a plurality of input signals. FIG. 3 illustrates an example of a perceptron constituting an artificial intelligence model applicable to the present invention. Referring to FIG. 3, a perceptron multiplies each input value (e.g., x=₁, x₂, x₃, . . . , xₙ) by weights 302-1 to 302-n (e.g., w₁ⱼ, w₂ⱼ, w₃ⱼ, . . . , wₙⱼ) and then adds up the weighted input values by using a transfer function 304. During the adding-up process, a bias value (e.g., bₖ) may be added. A perceptron generates an output value (e.g., oⱼ) by applying an activation function 406 for a net input value (e.g., netⱼ) that is an output of the transfer function 304. According to a case, the activation function 406 may operate based on a threshold (e.g., θⱼ). The activation function may be defined in various ways. A step function, a sigmoid, a Relu, and a Tanh may be used as an activation function, and the present invention is not limited thereto.

As illustrated in FIG. 3, an artificial neural network may be designed when perceptrons are arranged to form a layer. FIG. 4 illustrates an example of an artificial neural network constituting an artificial intelligence model applicable to the present invention. In FIG. 4, each node represented as a circle may be understood as a perceptron of FIG. 3. Referring to FIG. 4, an artificial neural network includes an input layer 402, a plurality of hidden layers 404a and 404b, and an output layer 406.

In case prediction is performed, when input data is provided to each node of the input layer 402, the input data is forward propagated to the output layer 406 through the input layer 402, weight application by perceptrons constituting the hidden layers 404a and 404b, a transfer function operation, and an activation function operation. On the other hand, in case training is performed, an error may be calculated through backward propagation from the output layer 406 to the input layer 402, and weights defined in each perceptron may be updated according to the calculated error.

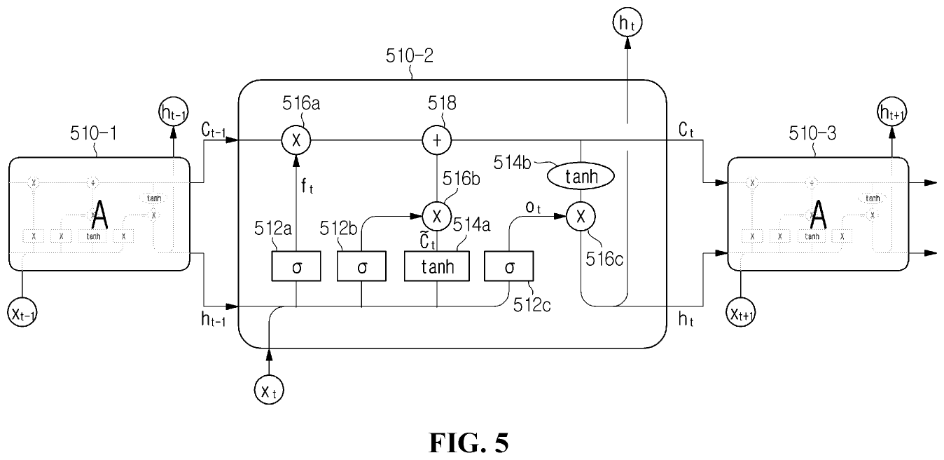

A recurrent neural network (RNN) is an artificial neural network, that is, a structure of determining a current state by using past input information. The RNN keeps using information, which is obtained in a previous step, by using an iterative structure. As a type of RNN, a long short-term memory (LSTM) network has been proposed. An LSTM network was proposed to control long-term dependency and has an iterative structure like RNN. Th LSTM network has a structure as in FIG. 5.

FIG. 5 illustrates an example of an LSTM network applicable to the present invention. Referring to FIG. 5, the LSTM network has a structure where hidden networks 510-1 to 510-3 are iterated between an input layer and an output layer. Accordingly, when inputs xₜ₋₁, xₜ, xₜ₊₁ and the like are provided over time, a hidden state value, which is output in the hidden network 510-1 for the input xₜ₋₁ at a time t−1, is input into the hidden network 510-2 for a next time t together with the input xₜ at the next time t. The hidden network 510-2 includes sigmoid networks 512a, 512b and 512c, tanh networks 514a and 514b, multiplication operators 516a, 516b and 516c, and an addition operator 518. Each of the sigmoid networks 512a, 512b and 512c has a weight and a bias and uses a sigmoid function as an activation function. Each of the tanh networks 514a and 514b has a weight and a bias and uses a sigmoid tanh function as an activation function.

The sigmoid network 512a functions as a forget gate. The sigmoid network 512a applies a sigmoid function to a weighted sum of a hidden state value hₜ₋₁ of a hidden layer of a previous time and input xₜ of a current time and then provides a result value as the multiplication operator 516a. The multiplication operator 516a multiplies the result value of the sigmoid function by a cell memory value Cₜ₋₁ of the previous time. Thus, the LSTM network may determine whether or not to forget a memory value of the previous value. That is, an output value of the sigmoid network 512a indicates how long the cell memory value Cₜ₋₁ of the previous time is to be maintained.

The sigmoid network 512b and the tanh network 514 function as an input gate. The sigmoid network 512b applies a sigmoid function to a weighted sum of a hidden state value hₜ₋₁ of a previous time t−1 and input xₜ of a current time t and then provides a result value iₜ to the multiplication operator 516b. The tanh network 514 applies a tanh function to a weighted sum of a hidden state value h t−1 of a previous time t−1 and input xₜ of a current time t and then provides a result value \tilde{C}ₜ to the multiplication operator 516b. The result value iₜ of the sigmoid network 512b and the result value \tilde{C}ₜ of the tanh network 514 are multiplied by the multiplication operator 516b and then are provided to the addition operator 510. Thus, the LSTM network may determine how much the input xₜ of a current time is to be reflected in the cell memory value Cₜ of a current time and then perform scaling according to determination. A cell memory value Cₜ₋₁ of a previous time, which is multiplied by a forget coefficient, and iₜ*\tilde{C}ₜ are added up by the addition operator 510. Thus, the LSTM network may determine the cell memory value Cₜ of the current time.

The sigmoid network 512c, the tanh network 514b, and the multiplication operator 516c function as an output gate. An output gate outputs a filtered value based on a cell state of a current time. The sigmoid network 512c applies a sigmoid function to a weighted sum of a hidden state value hₜ₋₁ of a previous time t−1 and input xₜ of a current time t and then provides a result value iₜ to the multiplication operator 516b. The tanh network 514b applies a tanh function to the cell memory value Cₜ of the current time t and then provides a result value to the multiplication operator 516c. The multiplication operator 516c generates a hidden state value h t of the current time t by multiplying a result value of the tanh network 514b and a result value of the sigmoid network 512c. Thus, the LSTM network may determine how long the cell memory value of the current time is to be maintained in a hidden layer.

In various disease systems, heterogeneity among patients may lead to different progression patterns and require different therapeutic interventions. Predicting desired outcomes from complex patient data is challenging due to temporal dynamics and heterogeneity of information. The LSTM network has been successfully used in various domains for processing sequential data. In particular, time-aware LSTM (T-LSTM) networks may process irregular time intervals within longitudinal patient records.

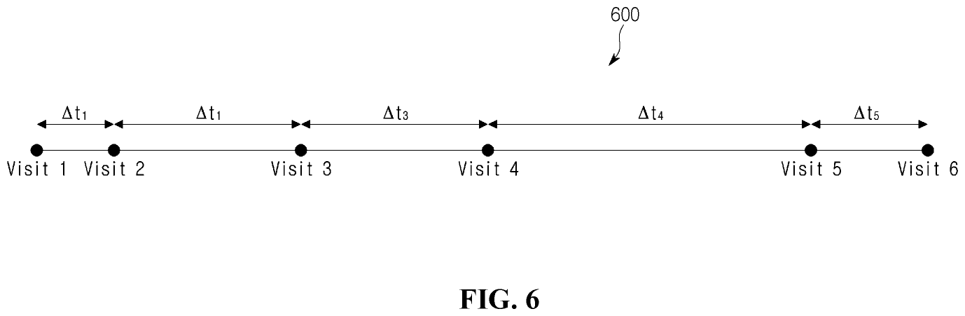

FIG. 6 illustrates an example of data used for predicting a disease onset possibility according to an embodiment of the present invention. FIG. 6 exemplifies data 600, indicating the time points of visits to an institution generating medical checkup results that can be used to predict disease onset possibility, that is, the time points at which medical checkups are conducted. Referring to FIG. 6, the data 600 shows a time interval between consecutive visits. Time intervals between two consecutive visits may vary and may span several years.

In the present invention, health examination or medical checkup means an act to obtain biometric data. Biometric information may include various information generated by the body, which may be obtained from user authentication elements (e.g., iris (retina), fingerprint, facial features), biometric signal elements (e.g., electrocardiogram (ECG), electromyography (EMG), electroencephalogram (EEG), electrooculogram (EOG), electroglottography (EGG), photoplethysmograph (Photo Plethysmo Graph, PPG), oxygen saturation (SpO2), blood sugar, cholesterol, blood flow), bioimpedance elements (e.g., GSR, body fat, body mass index (BMI), skin hydration, respiration), biomechanical elements (e.g., movement, joint relaxation, arterial blood pressure, pulse wave, heartbeat, vocal cord origin, respiratory sounds, heart sounds, blood flow, blood oxygenation, calorie consumption, body temperature, stress index, vascular age), or biochemical elements (e.g., urine, mucus, saliva, tears, blood, plasma, serum, sputum, cerebrospinal fluid, pleural fluid, nipple aspirate, lymphatic fluid, airway fluid, serous fluid, genitourinary tract fluid, breast milk, lymphatic fluid, semen, cerebrospinal fluid, tracheal fluid, ascites, cystic tumor fluid, amniotic fluid), and factors such as gender, age, height, weight, body size, family history, personal medical history, smoking habits, exercise habits, and alcohol consumption. In the present invention, medical checkup data, health checkup results, or checkup data may be understood as materials expressing biological information in numbers, letters, symbols, and the like.

Additionally, health data may be used apart from checkup data. Herein, health data refers to information related to the health of a corresponding person who is the subject for predicting diseases. According to various embodiments, health data may include at least one of general information, measurement information, blood information, and questionnaire information. For example, general information may include a person's age, gender, etc. For example, measurement information may include height, waist circumference as body indices and also include body mass index, blood pressure, etc. For example, blood information may include fasting blood sugar, total cholesterol, triglycerides, HDL cholesterol, LDL cholesterol, hemoglobin, serum creatinine, gamma-GTP, serum GOT, serum GPT, etc. For example, questionnaire information may include information written by the person themselves, such as family history, smoking, alcohol consumption, exercise information, etc.

In addition, health data may further include imaging information, genetic information, and life log information. For example, imaging information may include chest X-ray information obtained through chest X-ray examinations, electrocardiogram information obtained through electrocardiogram tests, and heart sound information related to vibrations caused by the closure of heart valves. For example, chest X-ray information is a picture of the inside of the chest using a very small amount of ionizing radiation to create a picture of the lungs, heart, and chest wall, which is used to evaluate the lungs, heart, and chest wall and may be used to diagnose various lung conditions such as shortness of breath, persistent cough, fever, chest pain, injury, pneumonia, emphysema, or cancer. For example, ECG information may be used to diagnose conditions of the heart, such as irregular rhythms or heart muscle damage. For example, heart sound information is information that quantifies measured heart sounds and converts them into an image represented by time on the horizontal axis and loudness on the vertical axis, which may be used to diagnose heart valve disease, etc. For example, genetic information is information about genes generated through genetic screening, which may be used to detect genetic variations and predict diseases caused by genetic variations. For example, life log information is information about blood pressure, body temperature, blood glucose levels, and the like that is recorded in a person's daily life via the terminal 40, such as a smartphone or wearable device owned by the person, and may be used to predict diseases and the like.

On the other hand, the health data may include health data corresponding to a plurality of times for a single person who is the subject of the disease prediction, and may also include time interval information between multiple times. In other words, each of the general information, measurement information, blood information, questionnaire information, imaging information, genetic information, and life log information included in the health data may be generated multiple times, and as a result, the health data may also include the time interval between the multiple times the health data was generated.

To overcome an irregular time interval between data, such as in FIG. 6, a system according to various embodiments may use a time aware (T)-LSTM network. A T-LSTM network has a structure capable of considering information about time intervals in reflecting a past state. In particular, in a T-LSTM network used in systems according to various embodiments, a last layer, that is, an output layer, has a structure designed to provide information about N times (e.g., N years). By using values corresponding to the N times as labels, a many-to-many method of the LSTM may be used to derive all the expected values up to a desired time. Such a structure has the advantage of being invariant to the number of visits.

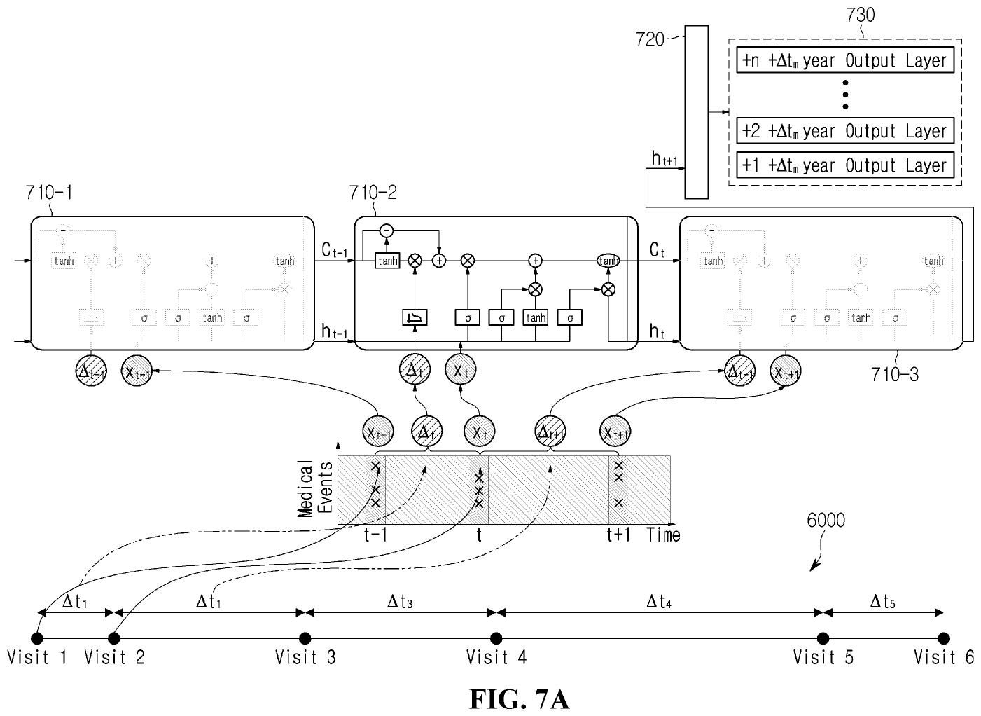

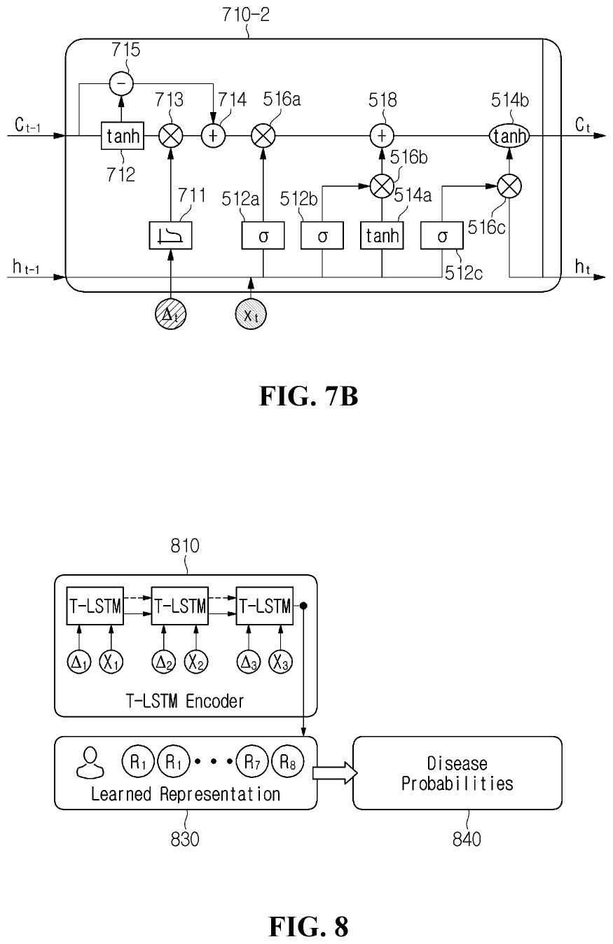

FIG. 7A illustrates an example of a structure of an artificial intelligence model for predicting a disease onset possibility according to an embodiment of the present invention. Referring to FIG. 7A, in the unevenly spaced data 6000, medical checkup data at each visit time (e.g., xₜ₋₁, xₜ, xₜ₊₁, etc.), and time interval values from a previous visit time (e.g., Δₜ₋₁, Δₜ, Δₜ₊₁, etc.) are provided to an AI model as input data. Herein, the medical checkup data includes information indicating whether or not given medical events occurred. For example, the medical checkup data may be a vector listing values associated with a given medical event, where each element of the vector may have a different format (e.g., binary value, measurement value, etc.) depending on the corresponding medical event. For example, for numerical data, specifically age, body mass index (BMI), fasting blood glucose levels, waist circumference, and various blood test results, the medical checkup data may include normalized values for each item in the overall population data, with the minimum value set to 0 and the maximum value set to 1. As another example, medical checkup data may include categorical data, specifically, data modeled with one-hot encoding, such as gender, family history, personal history, smoking status, exercise status, alcohol consumption, etc.

An artificial intelligence model has a structure in which hidden layers 710-1 to 710-3 are iterated. The hidden layer 710-1 for a time t−1 provides a cell memory value Cₜ₋₁ and a hidden state value h t−1 at the time t−1 to a hidden layer 710-1 of a next time t. Herein, a prediction result for a disease onset possibility may be generated from a hidden state value (e.g., hₜ₊₁) that is generated at a specific time. Specifically, the hidden state value hₜ₊₁ is input to an output vector generation layer 720, and a prediction result for a disease onset possibility is output from the output vector generation layer 720. The output vector generation layer 720 may have a fully connected layer form.

According to an embodiment, a prediction result is designed to have a vector form having onset possibility values for each of n years for a specific disease. Accordingly, the output layer 730, which outputs a prediction result, outputs a vector as long as the number of unit times (e.g., 1 year) that equally divide a predefined period (e.g., 10 years) and, to this end, the output layer 730 may be composed of as many nodes as the number of unit times. The structure and operation of the hidden layer 710-2 will be described in further detail with reference to FIG. 7B below.

FIG. 7B illustrates an example of a structure of a hidden layer of an artificial intelligence model for predicting a disease onset possibility according to an embodiment of the present invention. Referring to FIG. 7B, the hidden layer 710-2 for a time t receives a cell memory value Cₜ₋₁ and a hidden state value h t−1 at the time t−1 and generates a cell memory value Cₜ and a hidden state value h t at the time t. The hidden layer 710-2 includes a first network 711, a second network 712, a multiplication operator 713, an addition operator 714, a subtraction operator 715, sigmoid networks 512a, 512b and 512c, tanh networks 514a and 514b, multiplication operators 516a, 516b and 516c, and an addition operator 518. Herein, the function and operation of the sigmoid networks 512a, 512b and 512c, tanh networks 514a and 514b, multiplication operators 516a, 516b and 516c, and an addition operator 518 are the same as described with reference to FIG. 5.

The first network 711 uses a non-linear function as an activation function, The activation function of the first network 711 outputs a larger value from a smaller input value, that is, a time interval value Δₜ. When input value ranges are classified into a first range, a second range, and a third range, an absolute value of an input-to-output gradient in the first range may be larger than in the second range. That is, a change of an output value according to an increase of time interval in the first range may be larger than in the second range. In addition, an absolute value of an input-to-output gradient in the third range may be larger than in the second range. That is, the activation function of the first network 711 determines how much a state value of a previous time t−1 is to be reflected according to a degree of time interval.

The second network 712, the multiplication operator 713, the addition operator 714, and the subtraction operator 715 perform an operation to reflect a state value of the previous time t−1 as determined by the first network 711, that is, to an extent corresponding to an output of the first network 711. Specifically, the state value Cₜ₋₁ of the previous time t−1 is processed by the second network 712 that uses a tanh function as an activation function. In addition, the state value Cₜ₋₁ of the previous time t−1 is provided to the subtraction operator 715, and the subtraction operator 715 performs a subtracting operation between the state value Cₜ₋₁ and a result value of the second network 712. Herein, an output of the first network 711 may be referred to as a short-term memory value, and an output of the subtraction operator 715 may be referred to as a long-term memory value.

The multiplication operator 713 multiplies an output value of the second network 712 and an output value of the first network 711. That is, a short-term memory value is adjusted by using an output value of the first network 711 as a weight. Next, the addition operator 714 adds, that is, combines the weighted short-term memory value and the long-term memory value. Next, a combined value of the weighted short-term memory value and the long-term memory value is processed according to the operations that are described with reference to FIG. 5.

FIG. 8 illustrates an example of an output generated by an artificial intelligence model for predicting a disease onset possibility according to an embodiment of the present invention.

Referring to FIG. 8, prediction of a disease onset possibility may be performed by a recurrence operator 810 and a learned representation generator 830. The recurrence operator 810 has a structure in which a hidden layer is recurrently iterated. Each iteration generates cell memory values and hidden state values by using checkup result data at each time and a time interval value as inputs. A hidden state value of a last hidden layer may be input to the learned representation generator 820, and the learned representation generator 820 may determine a prediction result, that is, onset possibility information of a disease per unit time within a given period by reconstructing the input hidden state value.

According to the above-described various embodiments, an onset possibility of a disease by year may be predicted by using a T-LSTM network. In addition, a service according to various embodiments of the present invention may identify which factor has contributed to a prediction result for an onset possibility of a disease and may provide a corresponding result to a user. In order to identify a contributed factor for a prediction result, a layer-wise relevance propagation (LRP) technique may be used.

The LRP technique is helpful in verifying and understanding an accurate behavior of recurrent classifiers and may detect a main pattern in a text data set. In comparison with other non-gradient description schemes (e.g., those dependent on random sampling or iterative representation occlusion), this technique is deterministic and may be calculated as one pass through a network. Furthermore, since the LRP technique does not require any training of an external classifier to deliver description, the LRP technique is self-contained, and description is obtained directly from an original.

In a system according to various embodiments, the use of LRP is extended to recurrent neural networks (RNN). Since an increase of connection is caused in a recurrent network structure like LSTM, a specific forward propagation rule applicable to increasing connections may be redefined. According to an embodiment, in a 10-year prediction project on a yearly basis, the LRP technique may be applied to a word-based T-LSTM model. Thus, a reliable description may be provided regarding which word is responsible for contributing to factors in a patient record.

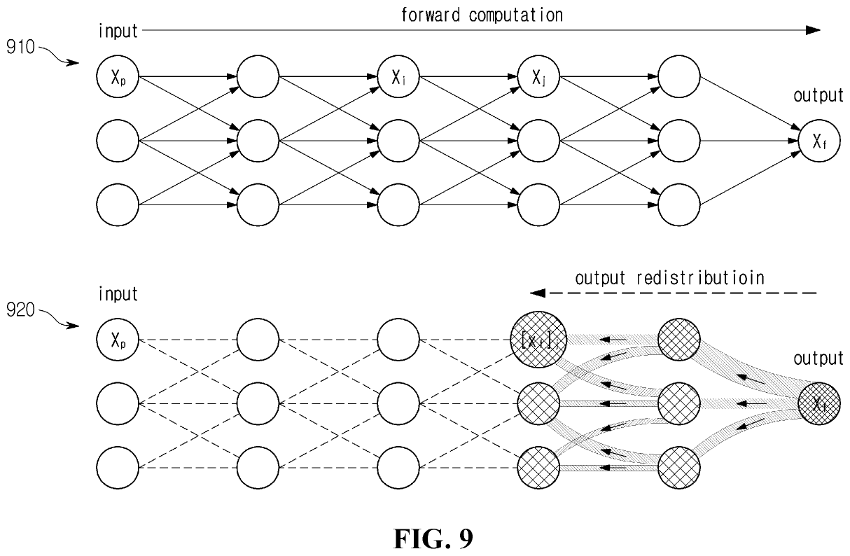

FIG. 9 illustrates a forward process for predicting a disease onset possibility and a reverse process for determining a contributed factor in accordance with an embodiment of the present invention. Referring to FIG. 9, a forward process 910 proceeds from an input layer to an output layer and generates a prediction result. On the other hand, a reverse process 910 proceeds from an output layer to an input layer and may determine factors contributing to a prediction result, which is generated by the forward process 910, by using the LRP technique.

The LRP technique according to various embodiments is based on a relevance conservation principle for each layer and redistributes a quantitative result (quantity fc(x)) by backpropagating the quantitative result from an output layer of a network to an input layer. An LRP relevance propagation procedure may be described according to each layer for each type of a layer generated in a deep convolutional neural network (CNN) and define a rule of giving a relevance to a lower layer neural by considering a relevance between upper layer neurons. Herein, each intermediate layer neuron may belong to a relevance score to an input layer neuron.

In a RNN structure like T-LSTM, the present invention restricts our definition about the LRP procedure to a many-to-one type. For convenience, the present invention does not explicitly provide a mark scheme for non-linear activation functions. If any activation exists in a neuron, the present invention may consider values of upper layer neurons that are activated in equations below. In order to calculate input space relevances, the present invention may start by setting a relevance of an output layer neuron corresponding to a target class c, which is interested in a value fc(x), and simply neglect other output layer neurons or set the relevance of the neurons to 0. Then, according to one of the following equations based on a type of related connection, the present invention may calculate a relevance score for each intermediate lower layer neuron according to each layer.

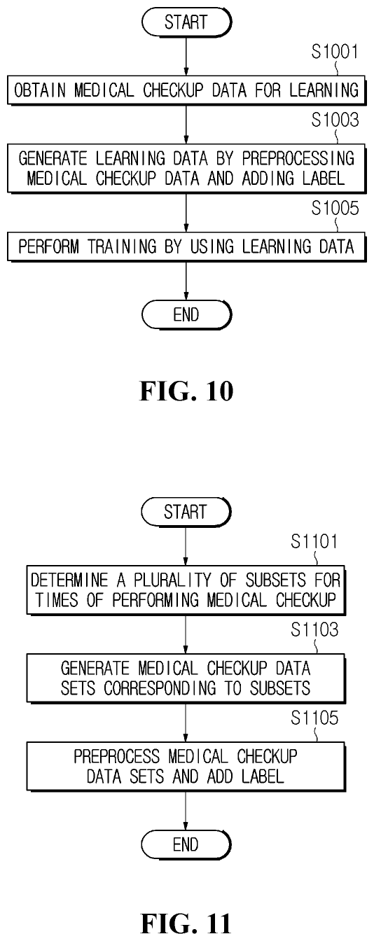

FIG. 10 illustrates an example of a procedure of training an artificial intelligence model according to an embodiment of the present invention. FIG. 10 exemplifies an operating method of a device with computing power (e.g., the service server 110 of FIG. 1).

Referring to FIG. 10, at step S1001, the device obtains medical checkup data for learning. The medical checkup data includes information on medical checkup results of a person (hereinafter, referred to as ‘examinee’) who had medical checkup in the past. Herein, the medical checkup data to be used for leaning includes information on a medical checkup result of at least one patient who is diagnosed with a target disease. In addition, the medical checkup data to be used for learning may further include information on a medical checkup result of a non-patient who has not been diagnosed with the target disease. Information on a medical checkup result may include information on a time (e.g., year) where medical checkup is conducted, and information on a checkup result that is obtained through medical checkup at each time. For example, medical checkup data for one patient may be as shown in Table 1 below.

TABLE 1

| | | Time | | |
|---|---|---|---|---|
| | | interval | | Disease |
| Examinee ID | Time (Year) | (Year) | Checkup result | diagnosis date |
| 0001 | 2003 | 0 | result_data_2003 | 2012 |
| 0001 | 2005 | 2 | result_data_2005 | |
| 0001 | 2009 | 4 | result_data_2009 | |

In Table 1, values belonging to the checkup result column may be defined in a different form according to a checkup item. At step S1003, the device generates learning data by preprocessing medical checkup data and adding a label. That is, the device processes the medical checkup data in a form available in an AI model and adds a label. Additionally, the device may remove examinee information (e.g., examinee ID) from medical checkup data. To this end, the device obtains the examinee's checkup result data for a specific disease and adds the checkup result data as a label. Herein, at step S1001, the checkup result data may be obtained together with the medical checkup data or be included in the medical checkup data. For example, the device allocates diagnosis result values of a disease to a unit time over a predetermined period (e.g., 10 years) from a latest year among times where checkup results included in medical checkup data are generated. Herein, among the diagnosis result values, a value during a period before the onset of disease is set to a value indicating normal, and a value after a time of the onset of the disease is set to a value indicating the onset of the disease. For example, when the examinee of Table 1 is diagnosed with a specific disease in 2012, a label may be as in Table 2 below.

TABLE 2

| Year | 2009 | 2010 | 2011 | 2012 | 2013 | 2014 | 2015 | 2016 | 2017 | 2018 |
|---|---|---|---|---|---|---|---|---|---|---|
| Value | 0 | 0 | 0 | 1 | 1 | 1 | 1 | 1 | 1 | 1 |

As shown in the example of Table 2, a start year of a label, that is, a base year is a latest year among times included in medical checkup data. That is, a label has a vector form including a value regarding whether or not a target disease occurs in each unit time (e.g., 1 year) that equally divides a predefined period (e.g., 10 years). At step S1005, the device performs training by using learning data. That is, the device inputs learning data into an AI model and performs back propagation based on a prediction result and a label, thereby updating at least one weight. In the example described with reference to FIG. 10, the device generates learning data by adding a label and performs training. Herein, for effective training, the device may augment learning data. In this case, the AI model may be trained by using basic learning data, which is generated based on medical checkup data, and augmented learning data that is generated based on data derived from the medical checkup data. An embodiment of augmentation of learning data is as in FIG. 11 below.

FIG. 11 illustrates an example of an augmentation procedure for learning data according to an embodiment of the present invention. FIG. 11 exemplifies an operating method of a device with computing power (e.g., the service server 110 of FIG. 1). In FIG. 11, medical checkup data of one examinee is described as an example. In case there are medical checkup data of a plurality of examinees, the procedure described below may be iteratively performed.

Referring to FIG. 11, at step S1101, a device determines a plurality of subsets for times of performing medical checkup. Specifically, the device generates at least one subset that combining at least one of the times of performing medical checkup, which is included in medical checkup data. For example, when medical checkup data including three times of the years 2003, 2005 and 2009, the at least one subset thus generated may include at least one of {2003}, {2005}, {2009}, {2003, 2005}, {2003, 2009}, and {2005, 2009}.

At step S1103, the device generates medical checkup data sets corresponding to subsets. Herein, the medical checkup data sets correspond to the subsets of the times respectively, and as many medical checkup sets as the number of subsets generated at step S1101 are generated. That is, the device may obtain new medical checkup data sets by combining checkup result information corresponding to times included in a subset and a subset of times. For example, from an original medical checkup data set as in Table 1 above, a medical checkup data set like at least one of Table 3 to Table 8 below may be obtained.

TABLE 3

| Examinee ID | Time (Year) | Time interval (Year) | Result |
|---|---|---|---|
| 0001 | 2003 | 0 | result_data_2003 |

TABLE 4

| Examinee ID | Time (Year) | Time interval (Year) | Result |
|---|---|---|---|
| 0001 | 2005 | 2 | result_data_2005 |

TABLE 5

| Examinee ID | Time (Year) | Time interval (Year) | Result |
|---|---|---|---|
| 0001 | 2009 | 4 | result_data_2009 |

TABLE 6

| Examinee ID | Time (Year) | Time interval (Year) | Result |
|---|---|---|---|
| 0001 | 2003 | 0 | result_data_2003 |
| 0001 | 2005 | 2 | result_data_2005 |

TABLE 7

| Examinee ID | Time (Year) | Time interval (Year) | Result |
|---|---|---|---|
| 0001 | 2003 | 0 | result_data_2003 |
| 0001 | 2009 | 6 | result_data_2009 |

TABLE 8

| Examinee ID | Time (Year) | Time interval (Year) | Result |
|---|---|---|---|
| 0001 | 2005 | 0 | result_data_2005 |
| 0001 | 2009 | 4 | result_data_2009 |

At step S1105, the device preprocesses medical checkup data sets and adds a label. That is, the device processes each medical checkup data set into a form available in an AI model and adds a label. Additionally, the device may remove information on an examinee (e.g., examinee ID) in each medical checkup data set. Accordingly, the device may obtain augmented learning data from one medical checkup data set. For example, learning data including at least one of [Table 9] to [Table 14] may further be obtained.

TABLE 9

| Medical | | | | | | | | | | |
|---|---|---|---|---|---|---|---|---|---|---|
| checkup | Checkup data | | | | | Time interval | | | | |
| data | result_data_2003 | | | | | 0 | | | | |
| Disease | 2003 | 2004 | 2005 | 2006 | 2007 | 2008 | 2009 | 2010 | 2011 | 2012 |
| diagnosis | 0 | 0 | 0 | 0 | 0 | 0 | 0 | 0 | 0 | 1 |
| label | | | | | | | | | | |

TABLE 10

| Medical | | | | | | | | | | |
|---|---|---|---|---|---|---|---|---|---|---|
| checkup | Checkup data | | | | | Time interval | | | | |
| data | result_data_2005 | | | | | 0 | | | | |
| Disease | 2005 | 2006 | 2007 | 2008 | 2009 | 2010 | 2011 | 2012 | 2013 | 2014 |
| diagnosis | 0 | 0 | 0 | 0 | 0 | 0 | 0 | 1 | 1 | 1 |
| label | | | | | | | | | | |

TABLE 11

| Medical | | | | | | | | | | |
|---|---|---|---|---|---|---|---|---|---|---|
| checkup | Checkup data | | | | | Time interval | | | | |
| data | result data 2009 | | | | | 0 | | | | |
| Disease | 2009 | 2010 | 2011 | 2012 | 2013 | 2014 | 2015 | 2016 | 2017 | 2018 |
| diagnosis | 0 | 0 | 0 | 1 | 1 | 1 | 1 | 1 | 1 | 1 |
| label | | | | | | | | | | |

TABLE 12

| Medical | Checkup data | | | | | Time interval | | | | |
|---|---|---|---|---|---|---|---|---|---|---|
| checkup | result_data_2003 | | | | | 0 | | | | |
| data | result_data_2005 | | | | | 2 | | | | |
| Disease | 2005 | 2006 | 2007 | 2008 | 2009 | 2010 | 2011 | 2012 | 2013 | 2014 |
| diagnosis | 0 | 0 | 0 | 0 | 0 | 0 | 0 | 1 | 1 | 1 |
| label | | | | | | | | | | |

TABLE 13

| Medical | Checkup data | | | | | Time interval | | | | |
|---|---|---|---|---|---|---|---|---|---|---|
| checkup | result_data_2005 | | | | | 2 | | | | |
| data | result_data_2009 | | | | | 4 | | | | |
| Disease | 2009 | 2010 | 2011 | 2012 | 2013 | 2014 | 2015 | 2016 | 2017 | 2018 |
| diagnosis | 0 | 0 | 0 | 1 | 1 | 1 | 1 | 1 | 1 | 1 |
| label | | | | | | | | | | |

TABLE 14

| | Checkup data | | | | | Time interval | | | | |
|---|---|---|---|---|---|---|---|---|---|---|
| Medical | result_data 2003 | | | | | 0 | | | | |
| checkup | result_data_2005 | | | | | 2 | | | | |
| data | result_data_2009 | | | | | 4 | | | | |
| Disease | 2009 | 2010 | 2011 | 2012 | 2013 | 2014 | 2015 | 2016 | 2017 | 2018 |
| diagnosis | 0 | 0 | 0 | 1 | 1 | 1 | 1 | 1 | 1 | 1 |
| label | | | | | | | | | | |

As described with reference to FIG. 11, a plurality of subsets may be extracted from times, and as many additional learning data sets as the number of extracted subsets may be obtained. According to an embodiment, the above-exemplified Table 9 to Table 14 may all be used as learning data. According to another embodiment, in augmentation of learning data, it is possible to apply a restriction that a time of medical checkup nearest to a time of diagnosing the onset of a disease should be included in a subset. In this case, among the above-exemplified Table 9 to Table 14, Table 9, Table 10 and Table 12, which do not include the year 2009, may be excluded from the data.

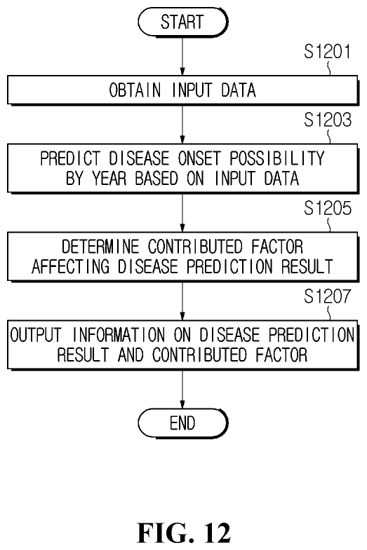

FIG. 12 illustrates an example of a procedure of predicting a disease onset possibility by using an artificial intelligence model according to an embodiment of the present invention. FIG. 12 exemplifies an operating method of a device with computing power (e.g., the service server 110 of FIG. 1).

Referring to FIG. 12, at step S1201, the device obtains input data. For example, the input data may be received from a client device (e.g., the client device 130 of FIG. 1). The input data may include medical checkup data of a subject, which is a target of predicting a disease onset possibility. Herein, the subject refers to a mammal which is suspected to undergo the onset of a disease or the recurrence of the disease or becomes an object for which examination is performed to see whether or not the disease has broken out or recurred. According to an embodiment, in order to use medical checkup data as input data, the device may preprocess the medical checkup data. In other words, the device may format the medical checkup data to be available as input data in an AI model. According to another embodiment, the formatting of the medical checkup data may be performed by a client device, and then the formatted data may be provided to the device.

At step S1203, the device predicts a disease onset possibility by year based on input data. To this end, the device generates output data indicating the disease onset possibility by year from the input data by using an AI model. The output data may be understood as a two-dimensional vector containing information on each disease and information on each year. That is, the output data may indicate which time (e.g., year) is likely to have the onset of each disease within a given period (e.g., 10 years) from now. For example, if it is the year 2021 now, the output data may be as shown in Table 15 below.

TABLE 15

| | 2021 | 2022 | 2023 | 2024 | 2025 | 2026 | 2027 | 2028 | 2029 | 2030 |
|---|---|---|---|---|---|---|---|---|---|---|
| Disease A | R_{A1} | R_{A2} | R_{A3} | R_{A4} | R_{A5} | R_{A6} | R_{A7} | R_{A8} | R_{A9} | R_{A10} |
| Disease B | R_{B1} | R_{B2} | R_{B3} | R_{B4} | R_{B5} | R_{B6} | R_{B7} | R_{B8} | R_{B9} | R_{B10} |
| . . . | . . . | . . . | . . . | . . . | . . . | . . . | . . . | . . . | . . . | . . . |

In Table 15, R_{A1} means a result value for a disease onset possibility at a first unit time for Disease A. According to an embodiment, the device may calculate a probability value for the disease onset possibility per unit time and provide probability values as outputs. In this case, RA1 is a probability value equal to or greater than 0 and equal to or less than 1. According to another embodiment, instead of the probability value, the device may provide binary values comparing the probability and a threshold. In this case, RA1 is a binary value indicating affirmation or negation (e.g., 1 or 0). At step S1205, the device determines a contributed factor affecting a disease prediction result. In other words, the device determines at least one item, which has a relatively large effect on a result of disease onset possibility by year obtained at step S1203, among various items included in the input data obtained at step S1201. For example, 10 items may be selected in descending order of effect. As another example, at least one item having contribution equal to or greater than a threshold level may be selected. Herein, nonadjustable factors, for example, family history, a subject's history, age and gender may be excluded from a selectable candidate pool. That is, at least one item may be selected from items that are subject to modification in future. To this end, the device may determine a relevance score of each node (e.g., perceptron) included in an AI model based on LRP technique sequentially from an output layer to an input layer. When a relevance score of nodes included in an input layer is calculated, the device selects some nodes based on the relevance score and checks input values corresponding to selected nodes. For example, the device may select nodes belonging to top n % of relevance scores or a node having a relevance score equal to or greater than a threshold. Factors corresponding to an input value thus checked are determined as an item that has a relatively large effect.

At step 1207, the device outputs information on a disease prediction result and a contributed factor. According to an embodiment, the device may generate data indicating the disease prediction result and the contributed factor and transmit the generated data to a client device. Accordingly, the client device may receive data, check a disease prediction result of a subject and a contributed factor based on the received data, and visualize (e.g., marking, output, etc.) or deliver (e.g., email, upload, etc.) to a subject.

According to an embodiment, a disease prediction method may be implemented by a recording medium including a program executed in a disease prediction system and/or computer.

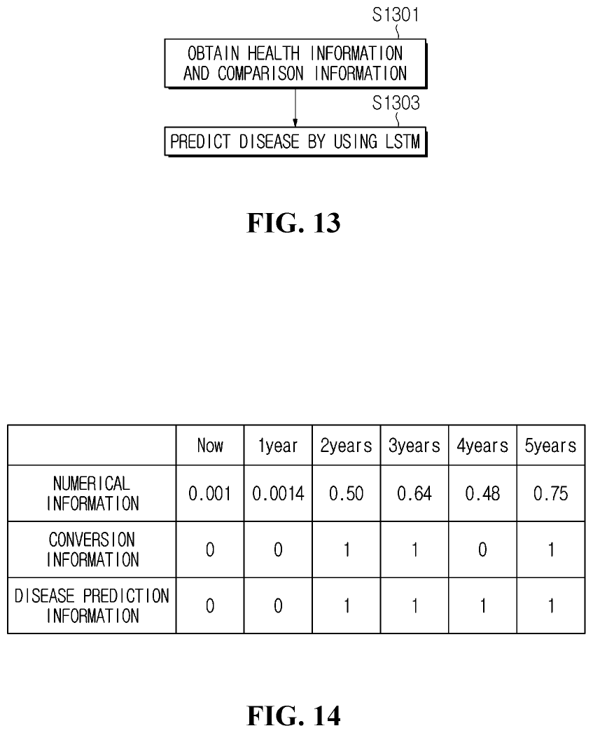

Referring to FIG. 13, a disease prediction method may include step S1301 where a communication unit (e.g., the communication unit 210 of FIG. 2) obtains health data of a person and comparison information from an external device. For example, the external device may include a server (e.g., the data server 120) of a medical institution such as a hospital, a server (e.g., the data server 120) of a public organization like National Health Insurance Service, and a terminal owned by a person (e.g., the client device 130).

According to an embodiment, step S1301 may include obtaining health data and comparison, which are basic data for predicting a person's disease, from an external device. For example, the communication unit may receive general information, measurement information, blood information, questionnaire information, imaging information, and genetic information from a server of a medical institution like hospital and obtain a creation time of each of the information. According to an embodiment, the communication unit may receive life log information from a person's terminal (e.g., the client device 130) and obtain a creation time of the information.

Herein, comparison information is information obtained from a server (e.g., the data server 120) of a public organization, for example, statistical data about people's health obtained from a server of National Health Insurance Service. According to an embodiment, the comparison information may include age-specific, age-based, and regional disease statistics, age-specific, age-based, and regional life expectancy, age-specific, age-based, and regional body index, age-specific, age-based, and regional obesity index, age-specific, age-based, and regional blood sugar index, age-specific, age-based, and regional cholesterol index, and other age-specific, age-based, and regional statistical information related to health. According to an embodiment, comparison information may be updated in a server of a public organization (e.g., the data server 120) every year, every three years, or every five years, and thus the comparison information may also include an updated time interval. Meanwhile, the comparison information is not limited to statistical data on the health of the public acquired from the server of the public organization (e.g., the data server 120) and, according to an embodiment, may include health data from multiple patients who have had a disease in the past and also include a time interval between the health data from the multiple patients who have had the disease.

According to an embodiment, the disease prediction method may include step S1303 where a processor calculates disease prediction information by using a long short-term memory (LSTM) based on health data and comparison information including a time interval. For example, the processor may predict a type of a disease and an onset time of the disease for a person who is a subject of disease prediction, based on the health data and comparison information obtained from an external device by the communication unit.

According to an embodiment, step S1303 may be implemented by machine learning using an LSTM. The LSTM is a type of recurrent neural network (RNN) and may be a machine learning program for analyzing current data by using previous data. According to an embodiment, health data about a person who is a subject of disease prediction may be generated multiple times (e.g., Visit 1 to Visit 6), and information on a time interval (e.g., Δt1 to Δt5) between the multiple times may also be generated. In addition, comparison information may also be updated multiple times, and an updated time interval between the multiple times may also be generated accordingly.

Herein, a processor may calculate disease prediction information by using two main types of data. The first type of data is a plurality of health data sets and data about comparison information, and the second type of data may include a time interval for a plurality of health data sets and/or a time interval for a plurality of pieces of comparison information. That is, the disease prediction method may predict a disease type and a disease onset time for a person who is a subject of disease prediction, more accurately by using, an input value, a reciprocal change of a plurality of health data sets, a reciprocal change of multiple pieces of comparison information, comparison between at least one health data set and at least one piece of comparison information and/or a time interval for a plurality of health data sets and/or a time interval for multiple pieces of comparison information.

Herein, according to an embodiment, step S1303 may calculate disease prediction information at a preset time interval from a present time to a future time, create numerical information quantifying an onset probability for a corresponding disease and, when the numerical information is equal to or greater than a preset threshold, determine that the disease occurs. An example of the numerical information is shown in FIG. 14. A disease prediction method according to an embodiment of the present invention is capable of providing a prediction result for a period of 10 years or longer, but FIG. 14 below shows a prediction result for a period of 5 years for convenience of description.

FIG. 14 illustrates an example of numerical information for explaining a step of producing disease prediction information in a method of predicting a disease according to an embodiment of the present invention. FIG. 14 exemplifies an example of data that is calculated by a processor, and the processor may create numerical information quantifying an onset probability of a specific disease for now and at a preset time interval respectively by operating health data and comparison information for a person who is a subject of disease prediction. The preset time interval may be defined by a user, but for convenience of description, one year is assumed in the following description. As illustrated in FIG. 14, numerical information for now may be 0.001, numerical information after 1 year from now may be 0.0014, and numerical information after 2 years from now may be 0.50.

Herein, according to an embodiment, when numerical information is equal to or greater than a preset threshold (e.g., 0.50), a processor may determine that a corresponding disease occurs. That is, numerical information for now and numerical information after 1 year from now may be equal to or less than a threshold of 0.50 and thus calculate disease prediction information for determining that a corresponding disease does not occur, and in this case, data of the disease prediction information may be set to a value of 0.

Meanwhile, numerical information after 2 years from now may be equal to or greater than 0.50 and thus calculate disease prediction information for determining onset of the disease. In this case, data of the disease prediction information may be set to a value of 1. That is, at step S1301, the processor may generate numerical information for a corresponding disease at a preset time interval from now and determine whether or not the disease occurs, based on whether or not the numerical information is equal to or greater than a preset threshold.

According to an embodiment, at step S1303, in case numerical information at a first time is equal to or greater than a preset threshold, even if numerical information at a second time, which is later than the first time, is less than the preset threshold, a corresponding disease may be determined to occur at the second time. More specifically, as illustrated in FIG. 14, the processor may generate numerical information on a corresponding disease at a preset time interval (e.g., 1 year) from now and generate conversion information by using the generated numerical information. For example, if the numerical information is equal to or greater than a preset criterion (e.g., 0.50), the conversion information may be set to 1, and if the numerical information is less than the preset criterion, the conversion information may be set to 0. Consequently, in case numerical information generated by year from now is 0.001, 0.0014, 0.50, 0.64, 0.48, and 0.75, conversion information by year from now to future may be determined as 0, 0, 1, 1, 0 and 1, respectively.

Herein, at step S1303, the processor may calculate disease prediction information regarding whether or not a corresponding disease occurs, based on the conversion information. Herein, according to an embodiment, in case the conversion information is a preset value (e.g., 1), the processor may define the disease prediction information as 1 to determine that the corresponding disease occurs, and in case the conversion information is not the preset value, the processor may define the disease prediction information as 0 to determine that the corresponding disease does not occur.

However, herein, as illustrated in FIG. 14, even if numerical information after 4 years from now is below a preset threshold, the processor may define the disease prediction information as 1 and calculate that the corresponding disease also occurs 4 years from now. More specifically, as illustrated in FIG. 14, when numerical information at a first time (e.g., after 2 years from now) is calculated as 0.50, as conversion information is determined as 1, the disease prediction information may be set to 1 to determine that the disease occurs. Herein, as numerical information at a second time (e.g., after 4 years from now) later than the first time is calculated as 0.48, although the conversion information is defined as 0, the disease prediction information is set to 1 so that the disease is calculated as onset.

That is, at step S1303, in case the conversion information is 0, the processor may calculate the disease prediction information as 0, and in case the disease prediction information is 1 at a previous time, the processor may calculate the disease prediction information as 1 even if the conversion information is 0. Consequently, by using numerical information, conversion information and disease prediction information, the processor may minimize an error of prediction result for a disease that is calculated by machine operation through an LSTM, and thus more accurate disease prediction information may be provided to a user.

According to the above-described various embodiments, a system may predict an onset possibility of a disease and provide information on a factor that greatly contributes to the prediction result. Using the above-described technique, an onset possibility of various diseases, such as various cancers, inflammatory diseases, autoimmune diseases, metabolic diseases, neurological diseases, and cardiovascular diseases, may be predicted within a predetermined period on a per-unit time basis (e.g., annually within a 10-year period from a most recent medical checkup).

The aforementioned various cancers include carcinoma, sarcoma, benign tumors, primary tumors, tumor metastasis, solid tumors, non-solid tumors, hematologic tumors, leukemia and lymphoma, and both primary and metastatic tumors. Carcinomas include esophageal carcinoma, hepatocellular carcinoma, basal cell carcinoma (e.g., in the form of skin cancer), squamous cell carcinoma (e.g., in various tissues), bladder carcinoma (e.g., including transitional cell carcinoma (e.g., malignant neoplasm of the bladder)), bronchogenic carcinoma, colonic carcinoma, colorectal carcinoma, gastric carcinoma, lung carcinoma (e.g., including small cell carcinoma and non-small cell carcinoma of the lung), adrenocortical carcinoma, thyroid carcinoma, pancreatic carcinoma, breast carcinoma, ovarian carcinoma, prostate carcinoma, sebaceous carcinoma, sweat gland carcinoma, sebaceous gland carcinoma, papillary carcinoma, papillary sebaceous carcinoma, cystadenocarcinoma, cholangiocarcinoma, renal cell carcinoma, intraductal carcinoma or bile duct carcinoma, mesothelioma, seminoma, embryonal carcinoma, Wilms tumor, cervical carcinoma, uterine carcinoma, testicular carcinoma, osteogenic carcinoma, epithelial carcinoma, and nasopharyngeal carcinoma, among others, but are not limited thereto.

Sarcomas include fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas, but are not limited thereto.

Solid tumors include neuroblastoma, germinoma, somatostatinoma, craniopharyngioma, pineal cell tumor, sertoli cell tumor, hemangiopericytoma, acoustic neuroma, lipoblastoma, meningioma, melanoma, ganglioneuroblastoma, and retinoblastoma, but are not limited thereto.

Leukemia includes a) chronic myeloproliferative syndromes (e.g., neoplastic disorders of pluripotent hematopoietic stem cells); b) acute myeloid leukemia (e.g., neoplastic transformation of pluripotent hematopoietic stem cells or hematopoietic cells with restricted lineage potential); c) chronic lymphocytic leukemia (CLL; clonal proliferation of immunologically immature and functionally incompetent small lymphocytes) (B-cell CLL, T-cell CLL, prolymphocytic leukemia, and hairy cell leukemia); and d) acute lymphoblastic leukemia (e.g., characterized by the accumulation of lymphoblasts), but is not limited thereto. Lymphoma includes B-cell lymphoma (e.g., Burkitt lymphoma) and Hodgkin lymphoma but is not limited thereto.

Benign tumors include, for example, hemangiomas, hepatocellular adenomas, capillary hemangiomas, focal nodular hyperplasia, acoustic neuromas, neurofibromas, bile duct adenomas, bile duct cystadenomas, fibromas, lipomas, leiomyomas, mesotheliomas, teratomas, myxomas, nodular regenerative hyperplasia, trachomas, and pyogenic granulomas, but are not limited thereto.

Primary and metastatic tumors may include, for example, lung cancer (including, but not limited to, lung adenocarcinoma, squamous cell carcinoma, large cell carcinoma, bronchioalveolar carcinoma, non-small cell carcinoma, small cell carcinoma, and mesothelioma); breast cancer (including, but not limited to, ductal carcinoma, lobular carcinoma, inflammatory breast cancer, clear cell carcinoma, and mucinous carcinoma); colorectal cancer (including, but not limited to, colon cancer and rectal cancer); pancreatic cancer (including, but not limited to, pancreatic ductal adenocarcinoma, acinar cell carcinoma, and neuroendocrine tumors); prostate cancer; ovarian cancer (including, but not limited to, ovarian epithelial carcinoma or surface epithelial-stromal tumors (including serous tumors), endometrioid tumors, and mucinous cystadenocarcinomas, sex cord-stromal tumors); liver and bile duct cancer (including, but not limited to, hepatocellular carcinoma, cholangiocarcinoma, and hemangioma); esophageal cancer (including, but not limited to, esophageal adenocarcinoma and squamous cell carcinoma); non-Hodgkin lymphoma; bladder cancer; uterine cancer (including, but not limited to, endometrial adenocarcinoma, uterine papillary serous carcinoma, uterine clear cell carcinoma, uterine sarcoma, and leiomyosarcoma, mixed Müllerian tumors); gliomas, astrocytomas, ependymomas, and other brain tumors; kidney cancer (including, but not limited to, renal cell carcinoma, clear cell carcinoma, and Wilms tumor); head and neck cancer (including, but not limited to, squamous cell carcinoma); stomach cancer (including, but not limited to, gastric adenocarcinoma, gastrointestinal stromal tumors); multiple myeloma; testicular cancer; germ cell tumors; neuroendocrine tumors; cervical cancer; carcinoids of the gastrointestinal tract, breast, and other organs; and chromophobe cell carcinoma. As specific examples, liver cancer, lung cancer, stomach cancer, colorectal cancer, breast cancer, prostate cancer, uterine cancer, thyroid cancer, and pancreatic cancer may be included.

The inflammatory disease refers to a disease that originates from inflammation, occurs from inflammation, or induces inflammation. The term “inflammatory disease” may also refer to a dysregulated inflammatory response caused by an excessive reaction from macrophages, granulocytes, and/or T-lymphocytes, which lead to abnormal tissue damage and cell death. In a specific example, an inflammatory disease includes an antibody-mediated inflammatory process. “Inflammatory disease” may be an acute or chronic inflammatory condition and may arise from an infectious or non-infectious cause Inflammatory diseases include, but are not limited to, atherosclerosis, arteriosclerosis, autoimmune disorders, multiple sclerosis, systemic lupus erythematosus, polymyalgia rheumatica (PMR), gouty arthritis, osteoarthritis, tendinitis, bursitis, psoriasis, cystic fibrosis, ankylosing spondylitis, rheumatoid arthritis, inflammatory arthritis, Sjogren's syndrome, giant cell arteritis, progressive systemic sclerosis (scleroderma), polymyositis, dermatomyositis, pemphigus, bullous pemphigoid, diabetes (e.g., type I), myasthenia gravis, Hashimoto's thyroiditis, Graves' disease, Goodpasture's disease, mixed connective tissue disease, sclerosing cholangitis, inflammatory bowel diseases, Crohn's disease, ulcerative colitis, aplastic anemia, inflammatory dermatoses, usual interstitial pneumonia (UIP), asbestosis, sarcoidosis, bronchiectasis, berylliosis, silicosis, coal worker's pneumoconiosis, lymphocytic interstitial pneumonia, granulomatous interstitial pneumonia, giant cell interstitial pneumonia, cellular interstitial pneumonia, extrinsic allergic alveolitis, Wegener's granulomatosis, and vasculitis-associated forms (temporal arteritis and polyarteritis nodosa), inflammatory dermatoses, hepatitis, delayed-type hypersensitivity (e.g., poison ivy), pneumonia, airway inflammation, adult respiratory distress syndrome (ARDS), encephalitis, immediate hypersensitivity, asthma, hay fever, allergies, acute anaphylaxis, rheumatic fever, glomerulonephritis, interstitial nephritis, epididymitis, cystitis, chronic cholecystitis, local anemia (ischemic injury), graft rejection, graft-versus-host rejection, appendicitis, arteritis, blepharitis, bronchiolitis, bronchitis, cervicitis, cholangitis, chorioretinitis, conjunctivitis, dacryoadenitis, dermatomyositis, endocarditis, endometritis, enteritis, episcleritis, epididymo-orchitis, fasciitis, connective tissue inflammation, gastritis, gastroenteritis, gingivitis, ileitis, iritis, laryngitis, meningitis, myocarditis, nephritis, orchitis, oophoritis, osteitis, otitis, pancreatitis, parotitis, pericarditis, pharyngitis, pleuritis, phlebitis, interstitial pneumonia, proctitis, prostatitis, rhinitis, salpingitis, sinusitis, stomatitis, synovitis, orchitis, tonsillitis, urethritis, urocystitis, uveitis, vaginitis, vasculitis, vulvitis, and balanitis, vasculitis, chronic bronchitis, osteomyelitis, optic neuritis, temporal arteritis, transverse myelitis, cerebral palsy-related fascilitis, and cerebral palsy-related enterocolitis.

The autoimmune diseases refer to the presence of autoimmune responses within an individual (immune responses acting against self-antigens or autoantigens). The autoimmune diseases include conditions that arise from the breakdown of self-tolerance, leading the adaptive immune system to respond against self-antigens and mediate cellular and tissue damage. In a specific example, an autoimmune disease is characterized, at least in part, as a result of a humoral immune response. Examples of autoimmune diseases include, but are not limited to, acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, antibody-mediated transplant rejection, anti-GBM/anti-TBM nephritis, antiphospholipid syndrome (APS), autoimmune angioedema, autoimmune aplastic anemia, autoimmune autonomic neuropathy, autoimmune hepatitis, autoimmune hyperlipidemia, autoimmune immunodeficiency, autoimmune inner ear disease (AIED), autoimmune myocarditis, autoimmune pancreatitis, autoimmune diabetic retinopathy, autoimmune thrombocytopenic purpura (ATP), autoimmune thyroid disease, autoimmune urticaria, axonal and neuron degeneration, Balo disease (Balo's concentric sclerosis), Behcet's disease, benign mucous membrane pemphigoid (cicatricial pemphigoid), cardiomyopathy, Castleman's disease, childhood adiposis dolorosa, Chagas disease, chronic fatigue syndrome, chronic inflammatory demyelinating polyneuropathy (CIDP), chronic recurrent multifocal osteomyelitis (CRMO), Churg-Strauss syndrome, cicatricial pemphigoid/benign mucous membrane pemphigoid, Crohn's disease, Cogan's syndrome, cold agglutinin disease, congenital heart block, coxsackie myocarditis, CREST syndrome (calcinosis, Raynaud's phenomenon, esophageal dysmotility, sclerodactyly, and telangiectasia), essential mixed cryoglobulinemia, demyelinating neuropathies, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, hypergammaglobulinemia, idiopathic thrombocytopenic purpura (ITP), IgA nephropathy, IgG4-related sclerosing disease, immune complex lipoprotein, inclusion body myositis, inflammatory bowel disease, insulin-dependent diabetes mellitus (type 1), interstitial cystitis, juvenile arthritis, juvenile diabetes, Kawasaki disease, Lambert-Eaton syndrome, leukocytoclastic vasculitis, lichen planus, lichen sclerosis, ligneous conjunctivitis, linear IgA disease (LAD), lupus (SLE), Lyme disease, Meniere's disease, microscopic polyangiitis, mixed connective tissue disease (MCTD), monoclonal gammopathy of undetermined significance (MGUS), Mooren's ulcer, MuSK antibody positive myasthenia gravis, multiple sclerosis, myasthenia gravis, myositis, narcolepsy, neuromyelitis optica (Devic's disease), neutropenia, ocular cicatricial pemphigoid, optic neuritis, palindromic rheumatism, PANDAS (pediatric autoimmune neuropsychiatric disorders associated with streptococcal infections), paraneoplastic cerebellar degeneration, paroxysmal nocturnal hemoglobinuria (PNH), progressive facial hemiatrophy, Parsonage-Turner syndrome, pars planitis (intermediate uveitis), pemphigoid, peripheral neuropathy, perivenous encephalomyelitis, pernicious anemia, POEMS syndrome, polyarteritis nodosa, polyglandular syndromes type I, II, and III (autoimmune), polymyalgia rheumatica, polymyositis, post-myocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reflex sympathetic dystrophy syndrome, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome (SPS), subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis (granulomatosis with polyangiitis (GPA)).

Metabolic diseases refer to a broad category of disorders caused by metabolic abnormalities within the body, specifically including obesity, type 1 diabetes, insulin-dependent diabetes, type 2 diabetes, hyperglycemia, dyslipidemia, obstructive sleep apnea, NAFLD (non-alcoholic fatty liver disease), NASH (non-alcoholic steatohepatitis), liver fibrosis, liver cirrhosis, hyperlipidemia, hypertension, atherosclerosis, and fatty liver, but are not limited thereto. In addition, the obesity may be a result of and/or related to metabolic abnormalities (e.g., hyperglycemia, hyperinsulinemia) and/or other factors (e.g., overeating, lack of physical exercise, etc.).

The neurological disorders may be selected from a group of Alzheimer's disease, Parkinson's disease, Huntington's disease, dementia, stroke, attention-deficit/hyperactivity disorder (ADHD), autism spectrum disorder (ASD), depression, bipolar disorder, schizophrenia, epilepsy, and multiple sclerosis (MS). The cardiovascular diseases include arrhythmia (e.g. atrial or ventricular or both), atherosclerosis and its sequelae, angina pectoris, cardiac rhythm disorders, myocardial ischemia, myocardial infarction, cardiac or vascular aneurysm, vasculitis, stroke, peripheral occlusive arterial disease, organ or tissue ischemia/reperfusion injury, shock state associated with significant drop in arterial blood pressure (e.g. septic, surgical, traumatic, or hypovolemic shock), pulmonary arterial hypertension (PAH), hypertension, cardiac valve disease, heart failure, blood pressure abnormalities, shock, vascular constriction (including those associated with migraines), vascular abnormalities, varicose vein therapy, renal or organ-limited failure, functional or organ venous insufficiency, cardiac hypertrophy, ventricular fibrosis, and myocardial remodeling.

The exemplary methods of the present invention are represented in a series of operations for clarity of description, but this is not intended to limit the order in which the steps are performed, and each step may be performed simultaneously or in a different order, if necessary. In order to realize a method according to the present invention, the steps illustrated may include further other steps, or may include the remaining steps with the exception of some steps, or may include additional other steps with the exception of some steps.

Various embodiments of the present invention are not intended to enumerate all possible combinations, but to describe a representative aspect of the present invention, and the matters described in the various embodiments may be applied independently or in combination of two or more.

In addition, various embodiments of the present invention may be realized by hardware, firmware, software, or a combination thereof. In the case of hardware realization, the embodiments may be realized by one or more ASICs (Application Specific Integrated Circuits), DSPs (Digital Signal Processors), DSPDs (Digital Signal Processing Digital Signal Processing Devices (DSPs), Programmable Logic Devices (PLDs), Field Programmable Gate Arrays (FPGAs), general processors, controllers, microcontrollers, microprocessors, etc.

The scope of the present invention includes software or machine-executable commands (e.g., operating systems, applications, firmware, programs, etc.) that allow an operation according to a method of various embodiments to be performed on a device or computer, and a non-transitory computer-readable medium in which such software or commands are stored and executed on the device or computer.

1. A method for predicting onset of a disease, the method comprising:
obtaining input data based on medical checkup data of a subject;
generating output data indicating an onset possibility of the disease by year from the input data by using a trained artificial intelligence model;
determining at least one item with a relatively high contribution to a result of the output data; and
outputting information regarding the onset possibility of the disease by year and the at least one item.

2. The method of claim 1, wherein the artificial intelligence model is trained by using learning data based on medical checkup data of at least one examinee diagnosed positive for the disease and at least one examinee diagnosed negative for the disease, and
wherein the learning data includes basic learning data generated based on the medical checkup data and augmented learning data generated based on data derived from the medical checkup data.

3. The method of claim 2, wherein the derived data includes data sets corresponding to a plurality of subsets for times of performing medical checkup included in the medical checkup data.

4. The method of claim 2, wherein the learning data includes a plurality of data sets,
wherein each of the plurality of data sets includes checkup result information of a first time, time difference information between a second time of performing the medical checkup immediately before the first time and the first time, and label data based on disease diagnosis time information of a corresponding examinee, and
wherein the label data has a vector form indicating whether or not the disease occurs per a unit time that equally divides a predefined period.

5. The method of claim 4, wherein the time difference information is set to 0, based on the first time being an earliest time of performing the medical checkup.

6. The method of claim 1, wherein the artificial intelligence model receives, as input, checkup result information of a subject for each time of a plurality of times and a time interval value from a previous time corresponding to each piece of the checkup result information, generates recurrently a hidden state value by considering the time interval value, and generates, as output, an onset possibility value of the disease per the unit time, which equally divides the predefined period, based on a final hidden state value that is generated by a predetermined number of cycles.

7. The method of claim 6, wherein the artificial intelligence model includes a network that generates output data in a form including as many onset possibility values of the disease as the number of unit times equally dividing the predefined period based on the final hidden state value.

8. The method of claim 1, wherein the determining of the at least one item comprises:
determining a relevance score of each node sequentially from an output layer to an input layer of the artificial intelligence model;
selecting at least one node among nodes in the input layer based on relevance scores of the nodes; and
checking at least one diagnosis item corresponding to the at least one selected node.

9. The method of claim 1, wherein the at least one item is selected from items that are subject to modification in future.

10. A method for predicting onset of a disease, the method comprising:
obtaining input data based on medical checkup data of a subject; and
providing output data indicating an onset possibility of the disease by year from the input data by using a trained artificial intelligence model,
wherein the artificial intelligence model is trained based on checkup result information of medical checkups performed at an unequal time interval, and
wherein the output data includes onset possibility values of the disease per a unit time that equally divides a predefined period.

11. A program stored on a medium to implement a method according to any one of claim 1 to claim 10 when operated by a processor.

12. A device for predicting onset of a disease, the device comprising:
a transceiver;
a storage unit configured to storing an artificial intelligence model; and
at least one processor coupled to the transceiver and the storage unit,
wherein the at least one processor is further be configured to:
obtain input data based on medical checkup data of a subject,
generate output data indicating an onset possibility of the disease by year from the input data by using a trained artificial intelligence model,
determine at least one item with a relatively high contribution to a result of the output data, and
output information regarding the onset possibility of the disease by year and the at least one item.

13. A device for predicting onset of a disease, the device comprising:
a transceiver;
a storage unit configured to storing an artificial intelligence model; and
at least one processor coupled to the transceiver and the storage unit,
wherein the at least one processor is further configured to:
obtain input data based on medical checkup data of a subject, and
provide output data indicating an onset possibility of the disease by year from the input data by using a trained artificial intelligence model,
wherein the artificial intelligence model is trained based on checkup result information of medical checkups performed at an unequal time interval, and
wherein the output data includes onset possibility values of the disease per a unit time that equally divides a predefined period.

14. A method of predicting a disease, the method comprising:
obtaining health data of a person and comparison information from an external device, wherein the health data includes health data of multiple times for the person and time interval data between the multiple times; and
calculating disease prediction information by using a long short-term memory (LSTM) based on the health data of the multiple times, the time interval data and the comparison information,
wherein the disease prediction information is calculated for future times that are allocated at a preset time interval from a present time,
wherein the disease prediction information is calculated based on numerical information that quantifies an onset probability for the disease corresponding to each of the times,
wherein the disease is determined to occur at the each of the times, based on the numerical information being equal to or greater than a preset threshold,
wherein, based on the numerical information being equal to or greater than the threshold at a first time among the times, even if the numerical information at a second time later than the first time is less than the preset threshold, the disease is also determined to occur at the second time,
wherein the time interval data between the multiple times includes a time interval value between adjacent multiple times,
wherein the time interval values are unequal,
wherein the health data includes, for the person, general information, measurement information, blood information, questionnaire information, imaging information, genetic information, and life log information, and
wherein the comparison information includes health data of a plurality of patients who have underwent the disease, and statistic data about health.
